(19) United States
(12) Patent Application Publication
(10) Pub. No.: US 2024/0285915 A1
(43) Pub. Date: Aug. 29, 2024

(54) INDEFLATION SYSTEM AND METHOD OF CONTROLLING THE INDEFLATION SYSTEM

(71) Applicant: TERUMO KABUSHIKI KAISHA, Tokyo (JP)

(72) Inventors: Kazuki NAKASONE, Ashigarakami-gun (JP); Kohei KANAYA, Fujisawa-shi (JP); Takumi FUKUDA, Hadano-shi (JP); Yoichiro KUWANO, Tokyo (JP)

(73) Assignee: TERUMO KABUSHIKI KAISHA, Tokyo (JP)

(21) Appl. No.: 18/441,450

(22) Filed: Feb. 14, 2024

(30) Foreign Application Priority Data
Feb. 28, 2023 (JP) 2023-030537

(51) Int. Cl. A61M 25/10; G06T 7/00; G06T 7/62

(52) U.S. Cl. CPC A61M 25/10184; G06T 7/0012; G06T 7/62; G06T 2207/10116; G06T 2207/30004

(57) ABSTRACT

An indeflation system includes a control unit configured to control an operation of a pump configured to pressurize or depressurize an inside of a catheter at a distal portion of which a balloon is provided, in which the control unit acquires a medical image of the balloon provided at the distal portion of the catheter inserted into a lumen, the balloon pressurized by the pump, detects presence or absence of an abnormality of the balloon on the basis of the medical image of the pressurized balloon, and controls the pump so as to depressurize the balloon in a case where the abnormality of the balloon is detected.

### CROSS-REFERENCES TO RELATED APPLICATIONS

This application claims priority to Japanese Application No. 2023-030537 filed on Feb. 28, 2023, the entire content of which is incorporated herein by reference.

### TECHNOLOGICAL FIELD

The present disclosure generally relates to an indeflation system and a control method of the indeflation system.

### BACKGROUND DISCUSSION

A balloon catheter is a catheter at a distal portion of which a balloon is provided. The balloon catheter is inserted into a patient body, and after the balloon is arranged or located at a target position, the balloon is inflated or deflated by pressurization or depressurization in the catheter. In general, the pressurization or depressurization in the catheter is performed by injecting or discharging an expansion fluid manually or by power drive using a pressurizer for an angioplasty balloon (indeflator). Japanese Patent Application Publication No. 2002-159578 A discloses a technology regarding inflation and deflation of a balloon catheter.

However, the conventional configuration can be improved in handling a case where an abnormality of the balloon such as rupture or slip (displacement) of the balloon occurs after the balloon catheter is arranged at a target position.

### SUMMARY

The present disclosure can more appropriately handle an abnormality of a balloon.

According to the present disclosure, an indeflation system (1) includes: a control unit configured to control an operation of a pump configured to pressurize or depressurize an inside of a catheter at a distal portion of which a balloon is provided, in which the control unit is configured to acquire a medical image of the balloon provided at the distal portion of the catheter inserted into a lumen, the balloon pressurized by the pump, detect presence or absence of an abnormality of the balloon on the basis of the medical image of the pressurized balloon, and control the pump so as to depressurize the balloon in a case where the abnormality of the balloon is detected.
(2) In the indeflation system according to (1), the control unit is configured to acquire the medical image of the balloon before being pressurized by the pump, determine whether a position of the balloon changes beyond a predetermined threshold between before and after pressurization of the pump on the basis of the medical image of the balloon before being pressurized and the medical image of the balloon after being pressurized, and detect the abnormality of the balloon in a case where it is determined that the position of the balloon changes beyond the threshold between before and after the pressurization of the pump.
(3) In the indeflation system according to (1) or (2), in which the control unit is configured to control the operation of the pump configured to pressurize or depressurize the inside of the catheter by moving a fluid containing a contrast agent in the catheter, and detect the abnormality of the balloon in a case where a brightness distribution of the medical image of the pressurized balloon changes beyond a predetermined threshold.
(4) In the indeflation system according to (3), in which the control unit is configured to specify, in the medical image of the pressurized balloon, a region including at least one of a portion where the balloon is located and a portion located downstream of the balloon in the lumen as a region of interest, and detect presence or absence of the abnormality of the balloon on the basis of a change in brightness distribution in the specified region of interest.
(5) In the indeflation system according to any one of (1) to (4), in which the control unit is configured to analyze the medical image of the pressurized balloon to measure a diameter of the balloon, and detect, in a case where the balloon is pressurized, when the diameter measured regarding the balloon does not change beyond a predetermined threshold or decreases beyond a predetermined threshold, the abnormality of the balloon.
(6) In the indeflation system according to any one of (2) to (5), in which the control unit is configured to acquire a pressure in the balloon, and detect, in a case where the balloon is pressurized, when a change rate of the pressure acquired regarding the balloon is smaller than a predetermined reference or the pressure acquired regarding the balloon decreases beyond a predetermined reference, the abnormality of the balloon using a smaller value as the predetermined threshold.
(7) In the indeflation system according to any one of (1) to (6), the pump configured to pressurize or depressurize the inside of the catheter at the distal portion of which the balloon is provided may further be included.

According to the present disclosure, a system (8) comprising: a balloon attached to a distal end of a catheter configured to be inserted into a luminal organ of a patient; a pump configured to inflate and deflate the balloon on the distal end of the catheter; and a control unit configured to: acquire a medical image of the balloon provided at the distal portion of the catheter inserted into a lumen, the balloon pressurized by the pump; detect presence or absence of an abnormality of the balloon on the basis of the medical image of the pressurized balloon; and control the pump so as to depressurize the balloon in a case where the abnormality of the balloon is detected.

According to the present disclosure, a control method of an indeflation system is (9) a control method of an indeflation system including a control unit configured to control an operation of a pump configured to pressurize or depressurize an inside of a catheter at a distal portion of which a balloon is provided, the control method including steps of: by the control unit, acquiring a medical image of the balloon provided at the distal portion of the catheter inserted into a lumen, the balloon pressurized by the pump; detecting presence or absence of an abnormality of the balloon on the basis of the medical image of the pressurized balloon; and controlling the pump so as to depressurize the balloon in a case where the abnormality of the balloon is detected.

According to one embodiment of the present disclosure, it is possible to more appropriately handle the abnormality of the balloon.

### BRIEF DESCRIPTION OF THE DRAWINGS

FIG. 1 is a block diagram illustrating a configuration example of an indeflation system according to one embodiment.

FIG. 2 is a view illustrating an example of an external appearance of an indeflation device and a balloon catheter in FIG. 1.

FIG. 3 is a view illustrating an example of an external appearance of an X-ray imaging device in FIG. 1.

FIG. 4 is a flowchart illustrating an operation example of the indeflation system.

FIG. 5 is a view illustrating an example of a medical image.

FIG. 6 is a flowchart illustrating an operation example of the indeflation system.

### DETAILED DESCRIPTION

Set forth below with reference to the accompanying drawings is a detailed description of embodiments of an indeflation system and a control method of the indeflation system. In the respective drawings, parts having the same configuration or function are denoted by the same reference sign. In the description of this embodiment, redundant description of the same part is sometimes appropriately omitted or simplified.

### Configuration of Indeflation System

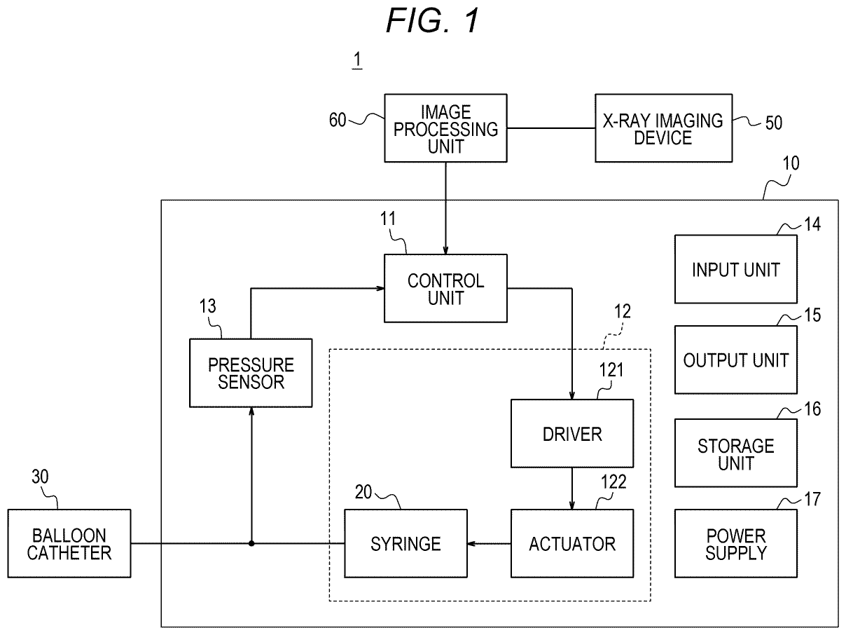

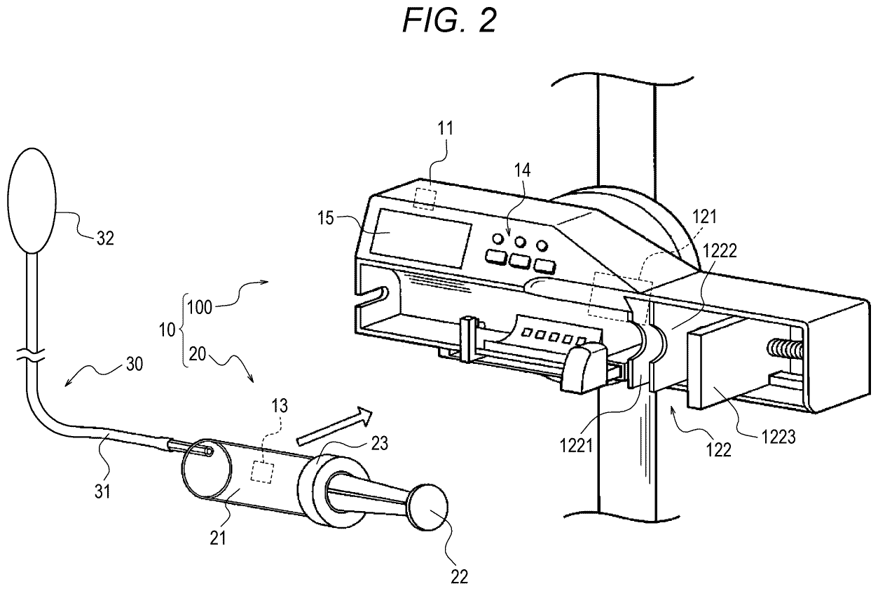

FIG. 1 is a block diagram illustrating a configuration example of an indeflation system (inflation/deflation system) 1 according to a first embodiment. The indeflation system 1 is provided with an indeflation device (inflation/deflation device) 10, a balloon catheter 30, an X-ray imaging device 50, and an image processing unit 60. FIG. 2 is a view illustrating an example of an external appearance of the indeflation device 10 and the balloon catheter 30 in FIG. 1. The indeflation device 10 pressurizes or depressurizes the inside of the balloon catheter 30 by moving a fluid in the balloon catheter 30. The balloon catheter 30 has a configuration in which a balloon 32 that inflates or deflates depending on an internal pressure is attached to a distal portion of a hollow catheter 31 (refer to FIG. 2). The balloon catheter 30 may be a disposable product that is discarded after one use. The X-ray imaging device 50 images the vicinity of the balloon 32 of a patient into which the balloon catheter 30 is inserted to acquire a medical image (angiographic image). The image processing unit 60 performs image processing on the medical image acquired by the X-ray imaging device 50 and detects an abnormality of the balloon 32. Hereinafter, a person who treats the patient using the indeflation system 1 is collectively referred to as a “user”. The user can be, for example, a medical worker including a doctor, a nurse or the like, and a plurality of persons may perform an operation as the users.

The indeflation device 10 is provided with a control unit 11, a pump 12, a pressure sensor 13, an input unit 14, an output unit 15, a storage unit 16, and a power supply 17. As illustrated in FIG. 2, in this embodiment, the indeflation device 10 can separate a syringe 20 from a device main body 100 having a configuration other than this. The indeflation device 10 is used in such a manner that the syringe 20 to which the catheter 31 of the balloon catheter 30 is attached is mounted on a housing unit of the device main body 100.

The control unit 11 includes one or more processors. In one embodiment, the “processor” is a general-purpose processor, or a dedicated processor specialized for specific processing, but is not limited to a general-purpose processor or a dedicated processor specialized for specific processing. The control unit 11 is communicably connected to each component that forms the indeflation device 10, and controls an operation of an entire indeflation device 10.

The pump 12 pressurizes or depressurizes the inside of the balloon catheter 30 by moving the fluid in the balloon catheter 30. The pump 12 is provided with a driver 121, an actuator 122, and the syringe 20.

The driver 121 as a drive unit provides a driving force required for moving the fluid. The driver 121 may be provided with, for example, a motor and the like.

The actuator 122 transmits the driving force provided by the driver 121 to the syringe 20. When the driver 121 drives after the actuator 122 is coupled with the syringe 20, a load is applied to a plunger 22 of the syringe 20 (refer to FIG. 2). In response to such application of the load, the plunger 22 moves in a longitudinal direction of a barrel 21 of the syringe 20. In the example in FIG. 2, the actuator 122 is provided with fixing members 1221 and 1222 and a movable portion 1223. The fixing members 1221 and 1222 are members that fix a blade 23 of the syringe 20 to be described later. The movable portion 1223 to which a proximal end side (a farther side from the balloon catheter 30) of the plunger 22 of the syringe 20 is attached moves in the longitudinal direction of the syringe 20 in response to the drive of the driver 121.

The syringe 20 is provided with the barrel 21 and the plunger 22. The barrel 21 stores the fluid, which is a medium for pressurizing or depressurizing the inside of the catheter 31. The fluid with which the barrel 21 is filled can be, for example, a liquid containing a contrast agent. By using the fluid containing the contrast agent, the user can perform an operation while confirming a position and a shape of the balloon catheter 30 in the imaged image in percutaneous coronary intervention in which display of the medical image imaged by the X-ray imaging device 50 is used together. As will be described later, in a case where the balloon 32 ruptures during the operation, the indeflation system 1 can recognize the abnormality of the balloon 32 by spread of the contrast agent in the medical image. The plunger 22 is provided in the barrel 21 filled with the fluid, and can move in the longitudinal direction of the barrel 21 to move the fluid. When the balloon catheter 30 is attached to the syringe 20, the barrel 21 and the inside of the balloon catheter 30 communicate with each other. The blade 23 is a protrusion provided on an outer periphery of the barrel 21. When the syringe 20 is housed in the housing unit of the device main body 100, the blade 23 is fixed between the fixing members 1221 and 1222. Accordingly, a position of the barrel 21 can be fixed with respect to the device main body 100. When the syringe 20 is housed in the housing unit of the device main body 100, the proximal end side of the plunger 22 of the syringe 20 is attached to the movable portion 1223 movable in the longitudinal direction of the syringe 20. Therefore, the driver 121 can pressurize the inside of the barrel 21 by moving the movable portion 1223 in a direction approaching the barrel 21. The driver 121 can depressurize the inside of the barrel 21 by moving the movable portion 1223 in a direction away from the barrel 21.

In this embodiment, the pump 12 is a syringe pump using the syringe 20, but the type of the pump 12 is not limited to a syringe pump. For example, the pump 12 may be a tube pump or a piston pump other than the syringe pump.

The pressure sensor 13 as a pressure detection unit measures a pressure of the fluid inside the barrel 21 and the balloon catheter 30. The pressure sensor 13 may be provided in the barrel 21 as illustrated in FIG. 2, or may be provided in another member such as the balloon catheter 30. Since the barrel 21 and the inside of the balloon catheter 30 communicate with each other, the pressure in the barrel 21 is the same as the pressure in the balloon catheter 30. A measurement value of the pressure in the balloon catheter 30 measured by the pressure sensor 13 is output to the control unit 11. The pressure sensor 13 measures the pressure with a pressure-sensitive element via a diaphragm (for example, a stainless diaphragm, a silicon diaphragm or the like), converts the pressure into an electric signal, and outputs the electric signal indicating the pressure to the control unit 11.

The input unit 14 includes one or more input interfaces that receive an input operation of the user and acquire input information based on the operation of the user. In this embodiment, the input unit 14 can be formed of a plurality of buttons or switches, but may be a touch screen provided integrally with a display of the output unit 15. Alternatively, the input unit 14 may be a physical key, a capacitance key, a pointing device, a microphone that receives a voice input or the like, but is not limited to a physical key, a capacitance key, a pointing device, or a microphone.

The output unit 15 includes one or more output interfaces that output information to the user and notify the user. For example, the output unit 15 is a display (display unit) that outputs the information as an image, and this may further be provided with a speaker and the like that outputs a voice. The output unit 15 may display, for example, the medical image imaged by the X-ray imaging device 50. Such display may be, for example, a liquid crystal panel display, an organic electro luminescence (EL) display or the like. At least one of the input unit 14 and the output unit 15 described above may be formed integrally with the indeflation device 10 or may be provided separately.

The storage unit 16 includes any storage module such as a solid state drive (SSD), a hard disk drive (HDD), a read-only memory (ROM), and a random access memory (RAM). The storage unit 16 may function as, for example, a main storage device, an auxiliary storage device, or a cache memory. The storage unit 16 stores any information used for the operation of the indeflation device 10. For example, the storage unit 16 may store various types of information used for controlling the pressure in the balloon catheter 30 in addition to a system program and an application program. The storage unit 16 may store the medical image imaged by the X-ray imaging device 50. The storage unit 16 is not limited to that built in the indeflation device 10, and may be an external database or an external storage module (for example, a universal serial bus (USB) memory and the like).

The power supply 17 supplies power to be a power source of the indeflation device 10. In this embodiment, the power supply 17 is formed of an AC adapter that is supplied with a steady alternating current (AC) from an external commercial power supply and converts the same into a direct current (DC) to be used in the indeflation device 10, but there is no limitation. For example, the power supply 17 may be formed of a battery such as a lithium ion battery.

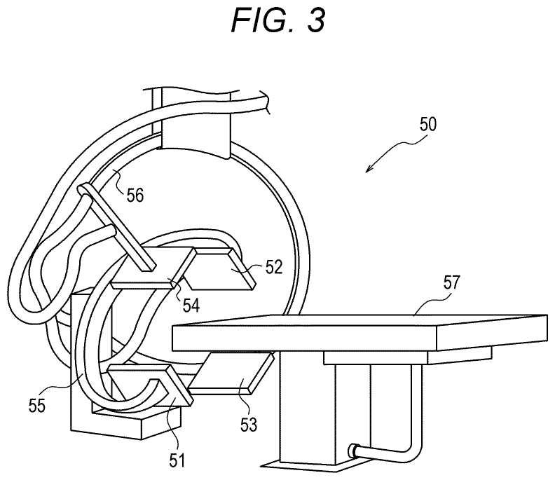

FIG. 3 is a view illustrating an example of an external appearance of the X-ray imaging device 50 in FIG. 1. The X-ray imaging device 50 irradiates a biological tissue such as the blood vessel into which the contrast agent is injected with an X-ray (radiation), detects the X-ray transmitted through the biological tissue, and acquires an angiographic image (also referred to as an angiographic image, an X-ray image, or a radiographic image) as the medical image. In this embodiment, the fluid for controlling the pressure of the balloon catheter 30 also contains the contrast agent, and the shape and the like of the balloon catheter 30 also appears in the medical image. As illustrated in FIG. 3, the X-ray imaging device 50 is provided with X-ray generation units 51 and 53, X-ray cameras 52 and 54, arms 55 and 56, and an inspection table 57. In this manner, the X-ray imaging device 50 is provided with two sets of the X-ray generation units 51 and 53 (i.e., two X-ray generation units) and the X-ray cameras 52 and 54 (i.e., two X-ray cameras), respectively, and can acquire two angiographic images imaged from different angles. In another embodiment, the X-ray imaging device 50 may be provided with one set of the X-ray generation unit (i.e., one X-ray generation unit) and the X-ray camera (i.e., one X-ray camera). The indeflation system 1 may be provided with any type of imaging device that images the medical image capable of detecting the abnormality of the balloon 32 in place of the X-ray imaging device 50.

The X-ray generation unit 51 irradiates the X-ray camera 52 with the X-ray. The X-ray camera 52 detects the X-ray applied from the X-ray generation unit 51 and generates the angiographic image. The arm 55 holds the X-ray generation unit 51 and the X-ray camera 52. The arm 55 can vary positions and directions of the X-ray generation unit 51 and the X-ray camera 52 with respect to the patient who is a subject while maintaining a relative positional relationship between the X-ray generation unit 51 and the X-ray camera 52 so that the X-ray camera 52 can receive the X-ray applied from the X-ray generation unit 51.

The X-ray generation unit 53 irradiates the X-ray camera 54 with the X-ray. The X-ray camera 54 detects the X-ray applied from the X-ray generation unit 53 and generates the angiographic image. The arm 56 holds the X-ray generation unit 53 and the X-ray camera 54. The arm 56 can vary positions and directions of the X-ray generation unit 53 and the X-ray camera 54 with respect to the patient while maintaining a relative positional relationship between the X-ray generation unit 53 and the X-ray camera 54 so that the X-ray camera 54 can receive the X-ray applied from the X-ray generation unit 53.

The inspection table 57 is a table on which the patient who is the subject lies. The X-ray generation unit 51 and the X-ray camera 52, and the X-ray generation unit 53 and the X-ray camera 54 form X-ray imaging units that acquire the angiographic image, respectively. The X-ray imaging device 50 transmits the angiographic images generated by the X-ray camera 52 and the X-ray camera 54 to the image processing unit 60 as the medical images. The X-ray imaging device 50 may continuously output an angiographic image of a moving image including a plurality of frames to the X-ray imaging device 50 while the indeflation system 1 is activated.

The image processing unit 60 performs image processing on the medical image received from the X-ray imaging device 50. Specifically, the image processing unit 60 performs processing for detecting the presence or absence of the abnormality of the balloon 32 by analyzing the medical image. The image processing unit 60 transmits the medical image received from the X-ray imaging device 50, an analysis result of the medical image and the like to the indeflation device 10. The indeflation device 10 displays the medical image, the analysis result and the like on the display unit of the output unit 15.

In the configuration as described above, the control unit 11 acquires the medical image of the balloon 32 inserted into a lumen and pressurized by the pump 12, and detects the presence or absence of the abnormality of the balloon 32 on the basis of the medical image. The abnormality of the balloon 32 can be, for example, rupture or slip (displacement) of the balloon 32. In a case where such abnormality of the balloon 32 is detected, the control unit 11 controls the pump 12 to depressurize the balloon 32. In this manner, since the indeflation system 1 automatically depressurizes the balloon 32 in a case where the abnormality of the balloon 32 is detected on the basis of the medical image of the balloon 32 being pressurized, it is possible to help prevent the inside of the lumen from being damaged by continuously pressurizing in a state in which the abnormality occurs in the balloon 32.

In this embodiment, the indeflation system 1 is provided with the indeflation device 10, the balloon catheter 30, the X-ray imaging device 50, and the image processing unit 60, and the indeflation device 10 is provided with the control unit 11, the pump 12, the pressure sensor 13, the input unit 14, the output unit 15, the storage unit 16, and the power supply 17, but the configuration is not limited to this. For example, the configuration provided with the pump 12 and the pressure sensor 13 may be configured as an independent device, and an operation of such device may be controlled by a general-purpose information processing device such as a personal computer (PC) provided with the control unit 11, the input unit 14, the output unit 15, and the storage unit 16. Alternatively, the indeflation device 10 may be provided with a part or all of the configurations of the X-ray imaging device 50 and the image processing unit 60. The control unit 11 may execute processing executed by the image processing unit 60.

In the example in FIG. 2, the pressure sensor 13 is provided in the barrel 21, but there is no limitation, and this may be formed integrally with the balloon catheter 30, for example. Furthermore, in the example in FIG. 2, the syringe 20 is detachable from the device main body 100, but may be formed integrally with the device main body 100.

### Operation Example 1

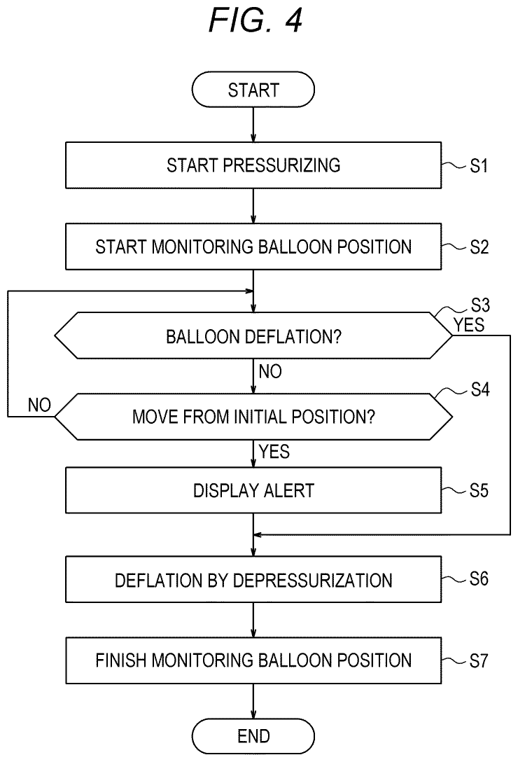

An operation of automatically depressurizing the balloon 32 in a case where the balloon 32 slips after the pressurization of the balloon catheter 30 is started will be described with reference to FIG. 4. FIG. 4 is a flowchart illustrating an operation example of the indeflation system 1. The operation of the indeflation system 1 described with reference to FIG. 4 may correspond to one of control methods of the indeflation system 1. An operation at each step in FIG. 4 can be executed on the basis of the control by the control unit 11 of the indeflation system 1.

Before the indeflation system 1 executes processing of the flowchart in FIG. 4, the user prepares for use of the indeflation system 1. Specifically, the user activates the indeflation device 10 and attaches the syringe 20 filled with the fluid to the device main body 100. The user attaches a required tool such as a tube of the balloon catheter 30 to the syringe 20, and performs a priming operation of removing air bubbles from a channel of the fluid so that air does not enter the balloon catheter 30. After completion of the priming operation, the user inserts the balloon catheter 30 into a body tube such as the blood vessel of the patient, and causes the balloon 32 to reach a target position such as a lesion while watching the medical image displayed on the display unit of the output unit 15. The user inputs necessary information such as a target value of the pressure (target pressure value) in the balloon catheter 30, and then instructs the indeflation device 10 to pressurize. In response to the instruction to pressurize from the user, the indeflation device 10 starts processing at S1 and subsequent steps.

At S1, the control unit 11 starts pressurizing the balloon catheter 30. Specifically, the control unit 11 transmits a control signal to the driver 121 to drive the driver 121 so that the plunger 22 moves to a distal end side (a side to which the balloon catheter 30 is coupled) of the barrel 21 in the barrel 21. When the plunger 22 moves to the distal end side of the barrel 21 in response to the drive of the driver 121, the fluid with which the barrel 21 is filled flows into the balloon catheter 30. In response to the inflow of the fluid into the balloon catheter 30, the pressure in the balloon catheter 30 increases and the balloon 32 inflates.

At S2, the control unit 11 starts monitoring a position of the balloon 32 in the medical image. For example, the control unit 11 may control the image processing unit 60 to recognize the position of the balloon 32 using a learning model acquired in advance by machine learning in the medical image acquired by the X-ray imaging device 50. Alternatively, the control unit 11 may control the image processing unit 60 to recognize the position of the balloon 32 by a marker attached to the balloon 32 in advance. The control unit 11 stores an initial position of the balloon 32 in the storage unit 16.

At S3, the control unit 11 determines whether to deflate the balloon 32. For example, the control unit 11 may determine to deflate the balloon 32 in a case where this receives an instruction to finish the processing from the user or a certain period of time elapses after the pressurization starts. In a case where the balloon 32 is deflated (YES at S3), the control unit 11 shifts to S6, and otherwise (i.e., the balloon is not deflated (NO at S3), this shifts to S4. The control unit 11 repeats the processing at S4 at predetermined time intervals (cycles) until it is determined at S3 that the balloon 32 is deflated.

At S4, the control unit 11 determines whether the balloon 32 moves from the initial position. Specifically, the control unit 11 acquires a current position of the balloon 32 from the image processing unit 60. The control unit 11 may determine that the balloon 32 moves from the initial position in a case where a distance between the current position of the balloon 32 and the initial position of the balloon 32 stored in the storage unit 16 is larger than a predetermined threshold. In a case where the balloon 32 moves from the initial position (YES at S4), the control unit 11 shifts to S5, and otherwise (i.e., the balloon 32 has not shifted from the initial position) (NO at S4), this returns to S3.

In a case where the balloon 32 moves from the initial position, the balloon 32 slips (displaces). If it is continuously pressurized in this manner, there is a possibility that the balloon 32 displaced from the lesion expands a normal blood vessel and causes damage such as blood vessel separation. Therefore, after S5, the control unit 11 warns the user and deflates the balloon 32.

At S5, the control unit 11 displays an alert (warning) indicating that the balloon 32 has slipped on the display unit of the output unit 15. Furthermore, the control unit 11 may output an alarm sound from a speaker of the output unit 15.

At S6, the control unit 11 depressurizes the balloon catheter 30 to deflate the balloon 32. Specifically, the control unit 11 transmits a control signal to the driver 121 to drive the driver 121 so that the plunger 22 moves to a proximal end side (a side to which the balloon catheter 30 is not coupled) of the barrel 21 in the barrel 21. When the plunger 22 moves to the proximal end side of the barrel 21 in response to the drive of the driver 121, the fluid in the balloon catheter 30 flows out to the barrel 21. In response to the outflow of the fluid from the balloon catheter 30, the pressure in the balloon catheter 30 decreases and the balloon 32 deflates. The control unit 11 may perform the alert display at S5 after the balloon 32 is deflated by the depressurization at S6.

At S7, the control unit 11 finishes monitoring the position of the balloon 32 in the medical image. When the processing at S7 is finished, the control unit 11 finishes the processing of the flowchart in FIG. 4.

In this manner, in Operation Example 1, the indeflation system 1 monitors the movement of the inflated balloon 32 imaged by the X-ray imaging device 50 by the image processing unit 60. In a case of detecting the movement of the inflated balloon 32, the indeflation system 1 transmits a command to the driver 121 to deflate the balloon 32. Therefore, according to the indeflation system 1, it is possible to help prevent the body lumen such as the blood vessel from being damaged by continuously pressurizing even though the balloon 32 slips.

### Operation Example 2

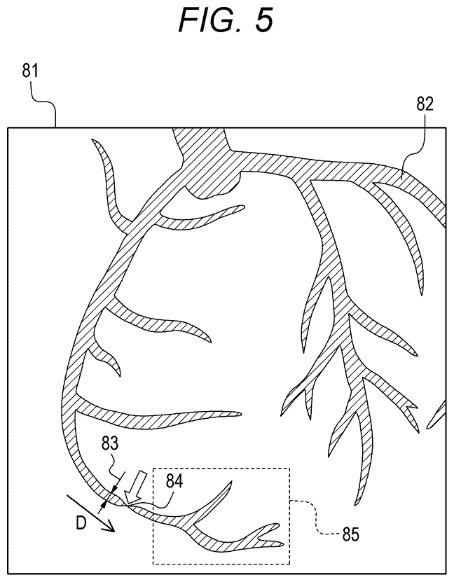

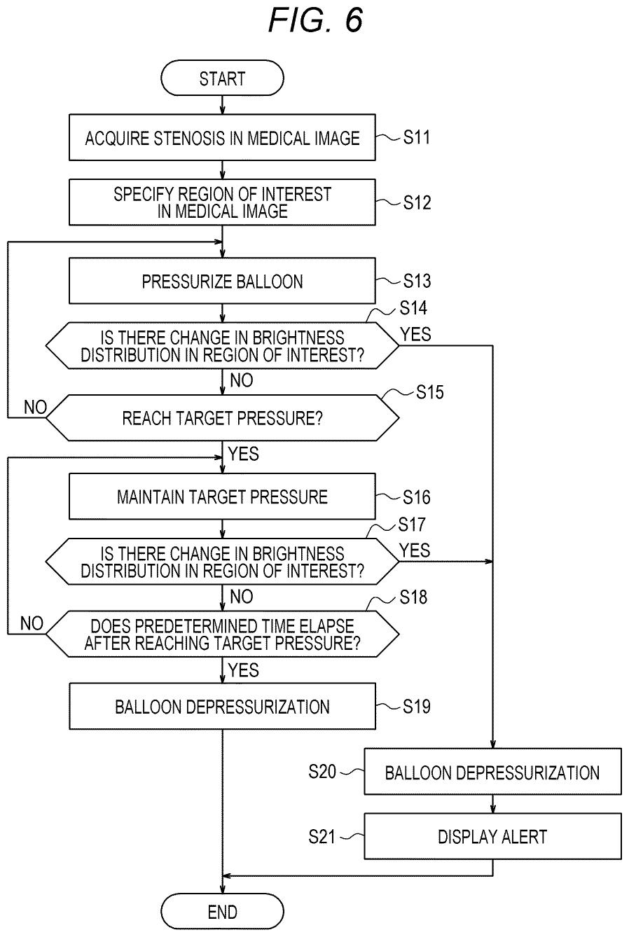

An operation of automatically depressurizing the balloon 32 in a case where the balloon 32 ruptures after the pressurization of the balloon catheter 30 is started will be described with reference to FIGS. 5 and 6.

FIG. 5 is a view illustrating an example of the medical image. In a medical image 81, a blood vessel region 82 is illustrated. When flowing the contrast agent in the blood flow, the blood vessel region 82 is displayed on the medical image 81 to be distinguished from other regions. In the example in FIG. 5, a stenosis 84 occurs on the blood vessel region 82. The stenosis 84 is a portion where a width 83 of the blood vessel region 82 is narrowed in the medical image 81. After the balloon 32 is positioned on such stenosis 84, the indeflation system 1 monitors a region 85 on a downstream side of the stenosis 84 as a region of interest after the pressurization is started. Here, in FIG. 5, a direction D indicates a direction of the blood flow.

There is a case where the balloon 32 is damaged by an excessive increase in pressure due to injection of the fluid of an allowable amount or more into the balloon 32, the presence of an inadvertent damage of the balloon 32, application of a stimulus from an external environment or the like. In such a case, it is desirable to immediately stop the pressurization of the balloon 32 and depressurize the balloon 32. When the balloon 32 ruptures, the contrast agent flows downstream of the stenosis 84 and a brightness distribution of the region 85 changes. Therefore, in a case where the brightness distribution of the region 85 changes beyond a predetermined threshold, the indeflation system 1 determines that there is the abnormality such as the rupture of the balloon 32 and depressurizes the balloon 32.

FIG. 6 is a flowchart illustrating an operation example of the indeflation system 1. The operation of the indeflation system 1 described with reference to FIG. 6 may correspond to one of control methods of the indeflation system 1. An operation at each step in FIG. 6 can be executed on the basis of the control by the control unit 11 of the indeflation system 1.

Before the indeflation system 1 executes processing of the flowchart in FIG. 6, the user prepares for use of the indeflation system 1; for example, the balloon 32 is arranged at a target position such as the stenosis 84. Details of the preparation for use are similar to those of Operation Example 1 described above.

At S11, the control unit 11 controls the image processing unit 60 to acquire the stenosis in the medical image. Specifically, as described with reference to FIG. 5, the image processing unit 60 may specify the blood vessel region 82 on the basis of the brightness of the medical image 81 and determine a portion where the width 83 of the blood vessel is smaller than a predetermined threshold as the stenosis 84. Alternatively, the image processing unit 60 may acquire the position of the balloon 32 in the medical image as the position of the stenosis. For example, similarly to S2 in FIG. 3, the image processing unit 60 may recognize the position of the balloon 32 using a learning model acquired in advance, a marker attached to the balloon 32 or the like. The image processing unit 60 notifies the control unit 11 of the acquired stenosis 84.

At S12, the control unit 11 controls the image processing unit 60 to specify the region of interest in the medical image. The region of interest is a region for the image processing unit 60 to determine the abnormality of the balloon 32 on the basis of the brightness distribution. As described with reference to FIG. 5, the image processing unit 60 may specify a certain region 85 on the downstream side of the stenosis 84 as the region of interest. Alternatively, the image processing unit 60 may specify a certain region including the stenosis 84 as the region of interest. For example, the image processing unit 60 may set a quadrangular or circular region centered around the stenosis 84 as the region of interest. The image processing unit 60 notifies the control unit 11 of the acquired region of interest. The control unit 11 may store the medical image in the specified region of interest in the storage unit 16.

At S13, the control unit 11 pressurizes the balloon 32. Specifically, the control unit 11 transmits a control signal to the driver 121 to drive the driver 121 so that the plunger 22 moves to a distal end side (a side to which the balloon catheter 30 is coupled) of the barrel 21 in the barrel 21. When the plunger 22 moves to the distal end side of the barrel 21 in response to the drive of the driver 121, the fluid with which the barrel 21 is filled flows into the balloon catheter 30. In response to the inflow of the fluid into the balloon catheter 30, the pressure in the balloon catheter 30 increases and the balloon 32 inflates.

At S14, the control unit 11 determines whether there is a change in brightness distribution in the region of interest specified at S12. Specifically, the control unit 11 may acquire a difference image regarding the brightness between the current medical image in the region of interest and the medical image stored in the storage unit 16 at S12. The control unit 11 may determine that there is the change in brightness distribution in a case where a total value of the brightness values of the respective pixels of the difference image exceeds a predetermined threshold. In a case where there is the change in brightness distribution in the region of interest (YES at S14), the control unit 11 shifts to S20, and otherwise (i.e., no change in brightness distribution in the region of interest) (NO at S14), this shifts to S15.

At S15, the control unit 11 determines whether the pressure in the balloon catheter 30 measured by the pressure sensor 13 reaches the target pressure. Specifically, in a case where the pressure in the balloon catheter 30 reaches the target value (YES at S15), the control unit 11 shifts to S16, and otherwise (NO at S15), this returns to S13. The control unit 11 repeats the processing at steps S13 and S14 at predetermined time intervals (cycles) until it is determined at S15 that the balloon 32 reaches the target pressure.

At S16, the control unit 11 controls the driver 121 so as to maintain the pressure in the balloon catheter 30 at the target pressure. At steps S16 to S18, the control unit 11 performs processing of maintaining the pressure in the balloon catheter 30 at the target pressure for a predetermined time. The predetermined time in which the target pressure is maintained may be, for example, a time instructed by the user or a time of a default value set in advance in the indeflation system 1.

At S17, the control unit 11 determines whether there is a change in brightness distribution in the region of interest specified at step S12. The control unit 11 can perform the processing at S17 as at S14. In a case where there is the change in brightness distribution in the region of interest (YES at S17), the control unit 11 shifts to step S20, and otherwise (i.e., no change in brightness distribution in the region of interest) (NO at S17), this shifts to S18.

At S18, the control unit 11 determines whether the predetermined time described above elapses after the pressure in the balloon catheter 30 reaches the target pressure. In a case where the predetermined time elapses (YES at S18), the control unit 11 shifts to S19, and otherwise (i.e., the predetermined time has not elapsed) (NO at S18), this returns to S16. The control unit 11 repeats the processing at S16 and S17 at predetermined time intervals (cycles) until it is determined at S18 that the predetermined time elapses.

At S19, the control unit 11 depressurizes the balloon 32. Specifically, the control unit 11 transmits a control signal to the driver 121 to drive the driver 121 so that the plunger 22 moves to the proximal end side of the barrel 21 in the barrel 21. When the plunger 22 moves to the proximal end side of the barrel 21 in response to the drive of the driver 121, the fluid in the balloon catheter 30 flows out to the barrel 21. In response to the outflow of the fluid from the balloon catheter 30, the pressure in the balloon catheter 30 decreases and the balloon 32 deflates. When the processing at S19 is finished, the control unit 11 finishes the processing of the flowchart in FIG. 6.

At S20, the control unit 11 depressurizes the balloon 32. The processing at S20 may be performed as at S19.

At S21, the control unit 11 displays an alert (warning) indicating that the balloon 32 rupture on the display unit of the output unit 15. Furthermore, the control unit 11 may output an alarm sound from a speaker of the output unit 15. The control unit 11 may deflate the balloon 32 by the depressurization at S20 after performing the alert display at S21. When the processing at S21 is finished, the control unit 11 finishes the processing of the flowchart in FIG. 6.

In this manner, in Operation Example 2, the indeflation system 1 monitors the brightness around the inflated balloon 32 (for example, the region on the downstream side of the balloon 32) imaged by the X-ray imaging device 50 by the image processing unit 60. In a case of detecting the change in brightness distribution, the indeflation system 1 transmits a command to the driver 121 to deflate the balloon 32. Therefore, according to the indeflation system 1, it is possible to help prevent the fluid from being delivered to the blood vessel and the like by continuously pressurizing even though the balloon 32 is damaged.

As described above, the indeflation system 1 is provided with the control unit 11 that controls the operation of the pump 12 that pressurizes or depressurizes the inside of the catheter 31 provided with the balloon 32 at the distal portion. The control unit 11 is provided at the distal portion of the catheter 31 inserted into the lumen and acquires the medical image of the balloon 32 pressurized by the pump 12. The control unit 11 detects the presence or absence of the abnormality of the balloon 32 on the basis of the medical image of the pressurized balloon 32, and controls the pump 12 to depressurize the balloon 32 in a case where the abnormality of the balloon 32 is detected.

In this manner, since the indeflation system 1 depressurizes the balloon 32 in a case where the abnormality of the balloon 32 is detected on the basis of the medical image of the balloon 32 being pressurized, it is possible to help prevent the inside of the lumen from being damaged.

The control unit 11 may acquire the medical image of the balloon 32 before being pressurized by the pump 12. On the basis of the medical image of the balloon 32 before being pressurized and the medical image of the balloon 32 after being pressurized, the control unit 11 may determine whether the position of the balloon 32 changes beyond a predetermined threshold between before and after the pressurization of the pump 12. The control unit 11 may detect the abnormality of the balloon 32 in a case where it is determined that the position of the balloon 32 changes beyond the threshold between before and after the pressurization of the pump 12.

In this manner, the indeflation system 1 detects the abnormality of the balloon 32 in a case where the change in position of the balloon 32 is determined between before and after the pressurization of the pump 12. Therefore, according to the indeflation system 1, it is possible to help prevent the inside of the lumen from being damaged due to continuous pressurization of the balloon 32 in a case where the balloon 32 slips in response to the pressurization.

The control unit 11 may control the operation of the pump 12 that pressurizes or depressurizes the inside of the catheter 31 by moving the fluid containing the contrast agent in the catheter 31. The control unit 11 may detect the abnormality of the balloon 32 in a case where the brightness distribution of the medical image of the pressurized balloon 32 changes beyond the predetermined threshold.

In this manner, in a case where the pump 12 pressurizes or depressurizes the inside of the catheter 31 by the movement of the fluid containing the contrast agent, the indeflation system 1 detects the presence or absence of the abnormality of the balloon 32 on the basis of the change in brightness distribution of the medical image. Therefore, according to the indeflation system 1, it is possible to effectively detect that the balloon 32 is damaged in the lumen and depressurize the balloon 32.

In the medical image of the pressurized balloon 32, the control unit 11 may specify the region 85 including at least one of the portion where the balloon 32 is located and the portion located on the downstream side of the balloon 32 in the lumen as the region of interest. The control unit 11 may detect the presence or absence of the abnormality of balloon 32 on the basis of the change in brightness distribution in the specified region of interest.

In this manner, the indeflation system 1 detects the presence or absence of the abnormality of the balloon 32 on the basis of the change in brightness distribution in the region of interest including at least one of the portion where the balloon 32 is located and the portion located on the downstream side of the balloon 32 in the lumen. Therefore, according to the indeflation system 1, it is possible to more effectively detect that the balloon 32 is damaged in the lumen.

Furthermore, the control unit 11 may measure a diameter of the balloon 32 by analyzing the medical image of the pressurized balloon 32. In a case where the balloon 32 is pressurized, the control unit 11 may detect the abnormality of the balloon 32 when the diameter measured regarding the balloon 32 does not change beyond a predetermined threshold or decreases beyond a predetermined threshold.

In this manner, when the diameter of the balloon 32 does not change or decreases despite of the pressurization, there is a relatively high possibility that the balloon 32 is damaged in the lumen. Therefore, by focusing on the diameter of the balloon 32, the indeflation system 1 can effectively detect the abnormality of the balloon 32 and depressurize the balloon 32.

Furthermore, the control unit 11 may detect the abnormality of the balloon 32 with reference to not only the medical image but also the pressure in the balloon catheter 30 detected by the pressure sensor 13.

Specifically, for example, the control unit 11 may acquire the pressure in the balloon 32, and detect, when a change rate of the pressure acquired regarding the balloon 32 is smaller than a predetermined reference or the pressure acquired regarding the balloon 32 decreases beyond a predetermined reference in a case where the balloon 32 is pressurized, the abnormality of the balloon 32 using a smaller value as the predetermined threshold. In this manner, when the change rate of the pressure within the balloon 32 is relatively small or the pressure decreases despite of the pressurization, there is a possibility that the balloon 32 has a damage of a pinhole (small hole). In such a case, by improving sensitivity of detecting the abnormality of the balloon 32 using a smaller threshold, the indeflation system 1 can effectively detect the abnormality of the balloon 32 and depressurize the balloon 32.

Alternatively, in a case where the balloon 32 that is expanding the stenosis 84 at a constant pressure slips, the balloon 32 moves to the normal blood vessel, so that constraint is released and the pressure inside the balloon 32 decreases. In a case where the balloon 32 that is expanding the stenosis 84 at a constant pressure ruptures, the pressure also decreases. Therefore, in a case of detecting such an unintended pressure decrease, the indeflation system 1 may determine that there is the abnormality of the balloon 32 and deflate the balloon 32. By performing such processing, the indeflation system 1 can help prevent damage to the normal blood vessel.

The present disclosure is not limited to the above-described embodiment. For example, a plurality of blocks illustrated in a block diagram may be integrated, or one block may be divided. Instead of being executed in a time-series manner according to the illustration, the plurality of steps illustrated in the flowchart may be executed in parallel or in a different order, depending on the processing capacity of the device that executes each step or as needed. In addition, modifications can be made without departing from the gist of the present disclosure.

The detailed description above describes embodiments of an indeflation system and a control method of the indeflation system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An indeflation system comprising:
a control unit configured to control an operation of a pump configured to pressurize or depressurize an inside of a catheter at a distal portion of which a balloon is provided; and
wherein the control unit is configured to:
acquire a medical image of the balloon provided at the distal portion of the catheter inserted into a lumen, the balloon pressurized by the pump;
detect presence or absence of an abnormality of the balloon on the basis of the medical image of the pressurized balloon; and
control the pump so as to depressurize the balloon in a case where the abnormality of the balloon is detected.

2. The indeflation system according to claim 1, wherein the control unit is configured to:
acquire the medical image of the balloon before being pressurized by the pump;
determine whether a position of the balloon changes beyond a predetermined threshold between before and after pressurization of the pump on the basis of the medical image of the balloon before being pressurized and the medical image of the balloon after being pressurized; and
detect the abnormality of the balloon in a case where it is determined that the position of the balloon changes beyond the threshold between before and after the pressurization of the pump.

3. The indeflation system according to claim 1, wherein the control unit is configured to:
control the operation of the pump configured to pressurize or depressurize the inside of the catheter by moving a fluid containing a contrast agent in the catheter; and
detect the abnormality of the balloon in a case where a brightness distribution of the medical image of the pressurized balloon changes beyond a predetermined threshold.

4. The indeflation system according to claim 3, wherein the control unit is configured to:
specify, in the medical image of the pressurized balloon, a region including at least one of a portion where the balloon is located and a portion located downstream of the balloon in the lumen as a region of interest; and
detect presence or absence of the abnormality of the balloon on the basis of a change in brightness distribution in the specified region of interest.

5. The indeflation system according to claim 1, wherein the control unit is configured to:
analyze the medical image of the pressurized balloon to measure a diameter of the balloon; and
detect, in a case where the balloon is pressurized, when the diameter measured regarding the balloon does not change beyond a predetermined threshold or decreases beyond a predetermined threshold, the abnormality of the balloon.

6. The indeflation system according to claim 2, wherein the control unit is configured to:
acquire a pressure in the balloon; and
detect, in a case where the balloon is pressurized, when a change rate of the pressure acquired regarding the balloon is smaller than a predetermined reference or the pressure acquired regarding the balloon decreases beyond a predetermined reference, the abnormality of the balloon using a smaller value as the predetermined threshold.

7. The indeflation system according to claim 1, further comprising:
the pump, the pump configured to pressurize or depressurize the inside of the catheter at the distal portion of which the balloon is provided.

8. A system comprising:
a balloon attached to a distal end of a catheter configured to be inserted into a luminal organ of a patient;
a pump configured to inflate and deflate the balloon on the distal end of the catheter; and
a control unit configured to:
acquire a medical image of the balloon provided at the distal portion of the catheter inserted into a lumen, the balloon pressurized by the pump;
detect presence or absence of an abnormality of the balloon on the basis of the medical image of the pressurized balloon; and
control the pump so as to depressurize the balloon in a case where the abnormality of the balloon is detected.

9. The system according to claim 8, wherein the control unit is configured to:
acquire the medical image of the balloon before being pressurized by the pump;
determine whether a position of the balloon changes beyond a predetermined threshold between before and after pressurization of the pump on the basis of the medical image of the balloon before being pressurized and the medical image of the balloon after being pressurized; and
detect the abnormality of the balloon in a case where it is determined that the position of the balloon changes beyond the threshold between before and after the pressurization of the pump.

10. The system according to claim 8, wherein the control unit is configured to:
control the operation of the pump configured to pressurize or depressurize the inside of the catheter by moving a fluid containing a contrast agent in the catheter; and
detect the abnormality of the balloon in a case where a brightness distribution of the medical image of the pressurized balloon changes beyond a predetermined threshold.

11. The system according to claim 10, wherein the control unit is configured to:
specify, in the medical image of the pressurized balloon, a region including at least one of a portion where the balloon is located and a portion located downstream of the balloon in the lumen as a region of interest; and
detect presence or absence of the abnormality of the balloon on the basis of a change in brightness distribution in the specified region of interest.

12. The system according to claim 8, wherein the control unit is configured to:
analyze the medical image of the pressurized balloon to measure a diameter of the balloon; and
detect, in a case where the balloon is pressurized, when the diameter measured regarding the balloon does not change beyond a predetermined threshold or decreases beyond a predetermined threshold, the abnormality of the balloon.

13. The indeflation system according to claim 9, wherein the control unit is configured to:
acquire a pressure in the balloon; and
detect, in a case where the balloon is pressurized, when a change rate of the pressure acquired regarding the balloon is smaller than a predetermined reference or the pressure acquired regarding the balloon decreases beyond a predetermined reference, the abnormality of the balloon using a smaller value as the predetermined threshold.

14. The indeflation system according to claim 9, further comprising:
a display configured to display an alert in the case where the abnormality of the balloon is detected.

15. A control method of an indeflation system including a control unit configured to control an operation of a pump configured to pressurize or depressurize an inside of a catheter at a distal portion of which a balloon is provided, the control method comprising:
acquiring, by the control unit, a medical image of the balloon provided at the distal portion of the catheter inserted into a lumen, the balloon pressurized by the pump;
detecting, by the control unit, presence or absence of an abnormality of the balloon on the basis of the medical image of the pressurized balloon; and
controlling, by the control unit, the pump so as to depressurize the balloon in a case where the abnormality of the balloon is detected.

16. The inflation method according to claim 15, further comprising:
acquiring, by the control unit, the medical image of the balloon before being pressurized by the pump;
determining, by the control unit, whether a position of the balloon changes beyond a predetermined threshold between before and after pressurization of the pump on the basis of the medical image of the balloon before being pressurized and the medical image of the balloon after being pressurized; and
detecting, by the control unit, the abnormality of the balloon in a case where it is determined that the position of the balloon changes beyond the threshold between before and after the pressurization of the pump.

17. The inflation method according to claim 15, further comprising:
controlling, by the control unit, the operation of the pump configured to pressurize or depressurize the inside of the catheter by moving a fluid containing a contrast agent in the catheter; and
detecting, by the control unit, the abnormality of the balloon in a case where a brightness distribution of the medical image of the pressurized balloon changes beyond a predetermined threshold.

18. The indeflation method according to claim 17, further comprising:
specify, by the control unit, in the medical image of the pressurized balloon, a region including at least one of a portion where the balloon is located and a portion located downstream of the balloon in the lumen as a region of interest; and
detecting, by the control unit, presence or absence of the abnormality of the balloon on the basis of a change in brightness distribution in the specified region of interest.

19. The indeflation method according to claim 15, further comprising:
analyzing, by the control unit, the medical image of the pressurized balloon to measure a diameter of the balloon; and
detecting, by the control unit, in a case where the balloon is pressurized, when the diameter measured regarding the balloon does not change beyond a predetermined threshold or decreases beyond a predetermined threshold, the abnormality of the balloon.

20. The indeflation method according to claim 16, further comprising:
acquiring, by the control unit, a pressure in the balloon; and
detecting, by the control unit, in a case where the balloon is pressurized, when a change rate of the pressure acquired regarding the balloon is smaller than a predetermined reference or the pressure acquired regarding the balloon decreases beyond a predetermined reference, the abnormality of the balloon using a smaller value as the predetermined threshold.
